(19) United States
(12) Patent Application Publication
(10) Pub. No.: US 2023/0229396 A1
(43) Pub. Date: Jul. 20, 2023

(54) APPARATUS AND METHOD WITH PARALLEL DATA PROCESSING

(71) Applicant: Samsung Electronics Co., Ltd., Suwon-si (KR)

(72) Inventors: Geuntae BAE, Seoul (KR); Keunmo PARK, Seoul (KR); Seungho LEE, Suwon-si (KR)

(73) Assignee: Samsung Electronics Co., Ltd., Suwon-si (KR)

(21) Appl. No.: 17/889,740

(22) Filed: Aug. 17, 2022

(30) Foreign Application Priority Data
Jan. 20, 2022 (KR) 10-2022-0008681

(51) Int. Cl. G06F 7/53; G06F 15/80; G06F 9/54

(52) U.S. Cl. CPC G06F 7/5324; G06F 15/8007; G06F 9/54

(57) ABSTRACT

An apparatus with parallel processing includes: a first processor module; and a second processor module configured to perform parallel processing in synchronization with the first processor module, wherein the first processor module is configured to: determine first operation result data using an operation process in a first time interval; transmit the first operation result data to the second processor module; determine second operation result data using the operation process in a second time interval; and determine whether to transmit the second operation result data to the second processor module, and wherein the second processor module is configured to determine second prediction result data corresponding to the second operation result data based on the first operation result data received from the first processor module and a prediction process in response to the first processor module determining not to transmit the second operation result data to the second processor module.

### CROSS-REFERENCE TO RELATED APPLICATIONS

This application claims the benefit under 35 USC § 119(a) of Korean Patent Application No. 10-2022-0008681, filed on Jan. 20, 2022 in the Korean Intellectual Property Office, the entire disclosure of which is incorporated herein by reference for all purposes.

### BACKGROUND

### 1. Field

The following description relates to an apparatus and method with parallel data processing.

### 2. Description of Related Art

A single task may be performed by tens to thousands of server clusters, based on large-scale distributed learning in the field of deep learning and a scientific computational simulation in various application fields, such as computational physics, computational chemistry, and biology. A molecular dynamics simulation, for example, is an application field of high performance computing (HPC) using parallel data processing, which simulates a change over time in multiple particles interacting in a two- or three-dimensional physical system through Newtonian mechanics. The molecular dynamics simulation may imitate a temporal change in the entire physical system by calculating a force applied to each of the particles over time and a consecutive position and movement speed of the particles. To perform the molecular dynamics simulation, an operation process on each of numerous particles and an operation process on interaction among the particles may be performed. For this, a large amount of data processing may be implemented. To effectively perform such data processing, a plurality of processors may be synchronized and perform data processing in parallel. When processors perform data processing in parallel for a single task, the processors may need to be synchronized with each other and exchange intermediate data.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect an apparatus with parallel processing includes: a first processor module; and a second processor module configured to perform parallel processing in synchronization with the first processor module, wherein the first processor module is configured to: determine first operation result data using an operation process in a first time interval; transmit the first operation result data to the second processor module; determine second operation result data using the operation process in a second time interval; and determine whether to transmit the second operation result data to the second processor module, and wherein the second processor module is configured to determine second prediction result data corresponding to the second operation result data based on the first operation result data received from the first processor module and a prediction process in response to the first processor module determining not to transmit the second operation result data to the second processor module.

For the determining of whether to transmit the second operation result data, the first processor module may be configured to determine not to transmit the second operation result data to the second processor module in response to a difference between the second operation result data and the first operation result data being less than a threshold.

For the determining of whether to transmit the second operation result data, the first processor module may be configured to: determine the second prediction result data based on the first operation result data and the prediction process; and determine not to transmit the second operation result data to the second processor module in response to a difference between the second operation result data and the second prediction result data determined by the first processor module being less than a threshold.

For the determining of whether to transmit the second operation result data, the first processor module may be configured to determine to transmit the second operation result data to the second processor module in response to a current number of transmissions to the second processor module satisfying a set condition.

The first operation result data and the second operation result data may be data in a floating point type, and the first processor module may be configured to transmit a mantissa part excluding an exponent part of the second operation result data to the second processor module in response to determining to transmit the second operation result data to the second processor module.

The first operation result data and the second operation result data may correspond to time series data, and the prediction process may include determining prediction result data using a Taylor series approximation technique approximating the time series data with a polynomial function.

The first processor module may be configured to: determine whether to transmit the first operation result data to the second processor module; and for the transmitting of the first operation result data, transmit the first operation result data to the second processor module in response to determining to transmit the first operation result data to the second processor module.

The second processor module may be configured to: determine whether the second prediction result data is determinable based on the received first operation result data and the prediction process and for the determining of the second prediction result data, determine the second prediction result data in response to the second prediction result data being determined to be determinable.

In response to determining to transmit the second operation result data to the second processor module, the first processor module may be configured to compress difference data between the second operation result data and the first operation result data and transmit the compressed difference data to the second processor module.

The second processor module may be configured to determine decompressed difference data by decompressing the compressed difference data and perform data restoration based on the decompressed difference data in response to receiving the compressed difference data from the first processor module.

The first processor module and the second processor module may be configured to operate movement of particles for a molecular dynamics simulation using the operation process.

The determining of the first operation result data using the operation process by the first processor module and the determining of the second prediction result data in a next time interval using the prediction process by the second processor module may be performed simultaneously in parallel.

In another general aspect, a processor-implemented method with parallel processing includes: determining first operation result data using an operation process by a first processor module in a first time interval; transmitting the first operation result data to a second processor module by the first processor module; determining second operation result data using the operation process by the first processor module in a second time interval; determining whether to transmit the second operation result data to the second processor module by the first processor module; and determining second prediction result data corresponding to the second operation result data based on the first operation result data received from the first processor module by the second processor module and a prediction process without transmitting the second operation result data to the second processor module by the first processor module in response to the first processor module determining not to transmit the second operation result data to the second processor module.

The determining of whether to transmit the second operation result data may include determining not to transmit the second operation result data to the second processor module in response to a difference between the second operation result data and the first operation result data being less than a threshold.

The determining of whether to transmit the second operation result data may include: determining the second prediction result data based on the first operation result data and the prediction process by the first processor module; and determining not to transmit the second operation result data to the second processor module in response to a difference between the second operation result data and the second prediction result data determined by the first processor module being less than a threshold.

The determining of whether to transmit the second operation result data may include determining to transmit the second operation result data to the second processor module in response to a current number of transmissions to the second processor module satisfying a set condition.

The first operation result data and the second operation result data may correspond to time series data, and the prediction process may include determining prediction result data using a Taylor series approximation technique approximating the time series data with a polynomial function.

The method may include determining whether the second prediction result data is determinable based on the first operation result data and the prediction process by the second processor module, wherein the determining of the second prediction result data by the second processor module may include determining the second prediction result data in response to the second prediction result data being determined to be determinable.

The method may include determining whether to transmit the first operation result data to the second processor module by the first processor module, wherein the transmitting of the first operation result data by the first processor module may include transmitting the first operation result data to the second processor module in response to determining to transmit the first operation result data to the second processor module.

The method may include: transmitting the second operation result data to the second processor module by the first processor module and determining the second prediction result data based on the first operation result data received from the first processor module by the second processor module and the prediction process in response to the first processor module determining to transmit the second operation result data to the second processor module; and receiving the second operation result data from the first processor module by the second processor module and transmitting the second prediction result data to another processor module, wherein the receiving of the second operation result data from the first processor module by the second processor module and the transmitting of the second prediction result data to the other processor module are performed simultaneously in parallel.

In another general aspect, one or more embodiments include a non-transitory computer-readable storage medium storing instructions that, when executed by one or more processors, configure the one or more processors to perform any one, any combination, or all operations and methods described herein.

In another general aspect, an apparatus with parallel processing includes: a processor module configured to perform parallel processing in synchronization with another processor module, and configured to: determine operation result data using an operation process in a time interval; and determine whether to transmit the operation result data to the other processor module based on a comparison of a threshold to a difference between the operation result data and another result data.

The other result data may be either one of: previous operation result data determined for a previous time interval; and prediction result data determined for the time interval based on the previous operation result data using a prediction process.

For the determining of whether to transmit the operation result data, the processor module may be configured to determine to transmit the operation result data in response to the difference being greater than or equal to the threshold.

The operation result data may correspond to a movement of particles in a border region between a first subspace and a second subspace of a simulation space.

Other features and aspects will be apparent from the following detailed description, the drawings, and the claims.

### BRIEF DESCRIPTION OF THE DRAWINGS

FIG. 1 illustrates an example of an overview of a parallel processing apparatus.

FIG. 2 illustrates an example of communication between processor modules.

FIGS. 3A and 3B illustrate an example of a predictor.

FIG. 4 illustrates an example of a communication method between processor modules in a parallel processing method.

FIGS. 5 and 6 illustrate an example of communication between processor modules.

FIG. 7 illustrates an example of a communication method between processor modules in a parallel processing method.

FIG. 8 illustrates an example of communication between processor modules.

FIG. 9 illustrates an example of a communication method between processor modules in a parallel processing method.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known, after an understanding of the disclosure of this application, may be omitted for increased clarity and conciseness.

Although terms such as “first,” “second,” and “third” are used to explain various members, components, regions, layers, or sections, these members, components, regions, layers, or sections are not to be limited by these terms. Rather, these terms should be used only to distinguish one member, component, region, layer, or section from another member, component, region, layer, or section. For example, a “first” member, component, region, layer, or section referred to in the examples described herein may also be referred to as a “second” member, component, region, layer, or section without departing from the teachings of the examples.

Throughout the specification, when a component is described as being “connected to,” “coupled to”, or “accessed to” another component, it may be directly “connected to,” “coupled to”, or “accessed to” the other component, or there may be one or more other components intervening therebetween. In contrast, when an element is described as being “directly connected to,” “directly coupled to”, or “directly accessed to” another element, there can be no other elements intervening therebetween. Likewise, similar expressions, for example, “between” and “immediately between,” and “adjacent to” and “immediately adjacent to,” are also to be construed in the same way. As used herein, the term “and/or” includes any one and any combination of any two or more of the associated listed items.

The terminology used herein is for describing various examples only and is not to be used to limit the disclosure. The articles “a”, “an”, and “the” are intended to include the plural forms as well, unless the context clearly indicates otherwise. As used herein, the term “and/or” includes any one and any combination of any two or more of the associated listed items. As used herein, the terms “include,” “comprise,” and “have” specify the presence of stated features, numbers, operations, elements, components, and/or combinations thereof, but do not preclude the presence or addition of one or more other features, numbers, operations, elements, components, and/or combinations thereof. The use of the term “may” herein with respect to an example or embodiment (for example, as to what an example or embodiment may include or implement) means that at least one example or embodiment exists where such a feature is included or implemented, while all examples are not limited thereto.

Unless otherwise defined, all terms used herein including technical or scientific terms have the same meanings as those generally understood consistent with and after an understanding of the present disclosure. Terms, such as those defined in commonly used dictionaries, should be construed to have meanings matching with contextual meanings in the relevant art and the present disclosure, and are not to be construed as an ideal or excessively formal meaning unless otherwise defined herein.

Hereinafter, examples will be described in detail with reference to the accompanying drawings. When describing the examples with reference to the accompanying drawings, like reference numerals refer to like components and a repeated description related thereto will be omitted.

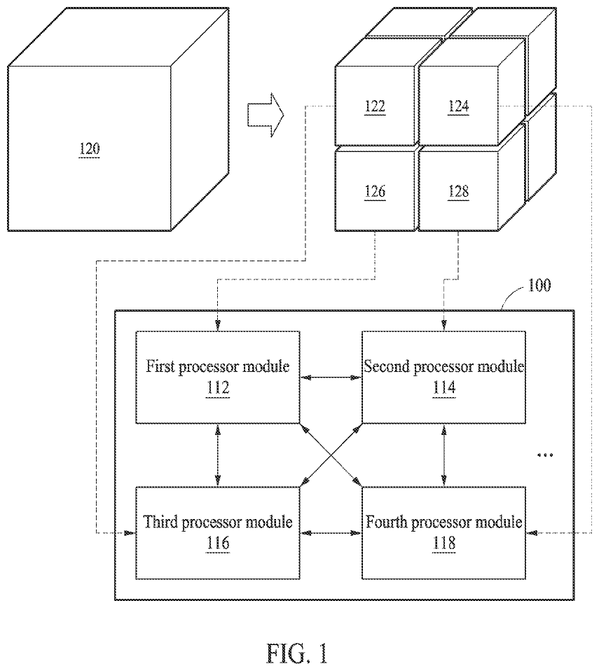

FIG. 1 illustrates an example of an overview of a parallel processing apparatus.

Referring to FIG. 1, a parallel processing apparatus 100 is an apparatus for performing parallel processing and may be used for high performance computing (HPC) for performing a parallel operation using a large number of operation resources. For example, the parallel processing apparatus 100 may be used for multi-node distributed computing including multiple server resources in a form of a cluster on a network to rapidly perform a molecular dynamics simulation involving a large amount of operation processes. In addition, the parallel processing apparatus 100 may be applicable to an application involving a large-scale operation, such as deep learning, a weather forecasting simulation, a scientific computational simulation, a physical property analysis, and/or the like.

The parallel processing apparatus 100 may include a plurality of processor modules, for example, first, second, third, and fourth processor modules 112, 114, 116, and 118 for performing an operation. In an example, four processor modules (e.g., the first, second, third, and fourth processor modules 112, 114, 116, and 118) are illustrated for ease of description, but an example may not be limited thereto. The parallel processing apparatus 100 may include two or more processor modules for performing an operation, and the number of processor modules is not limited thereto. For example, the parallel processing apparatus 100 may further include a central processor module for controlling each of the first, second, third, and fourth processor modules 112, 114, 116, and 118.

Each of the first, second, third, and fourth processor modules 112, 114, 116, and 118 may perform a parallel operation for a given task and perform parallel processing in synchronization with one or more other processor modules. For example, the second processor module 114 may perform parallel processing in synchronization with the first processor module 112. Each of the first, second, third, and fourth processor modules 112, 114, 116, and 118 may be or include a hardware processor (e.g., one or more processors) such as any one or any combination of any two or more of a central processing unit (CPU), a graphics processing unit (GPU), a neural processing unit (NPU), and a field programmable gate array (FGPA).

In an example, when the parallel processing apparatus 100 performs an operation for a molecular dynamics simulation, the parallel processing apparatus 100 may simulate a change over time in multiple particles interacting in a three-dimensional system 120 through Newtonian mechanics. The parallel processing apparatus 100 may perform parallel processing using the first, second, third, and fourth processor modules 112, 114, 116, and 118 to perform a large amount of operations more rapidly. The first, second, third, and fourth processor modules 112, 114, 116, and 118 may perform parallel processing for dividing a task and simultaneously processing the divided task and use a space division method for the parallel processing. The space division method may be a method of dividing the three-dimensional system 120 corresponding to an entire simulation space by the number of operation resources (e.g., processor modules) and simultaneously processing each of the divided space (e.g., first, second, third, and fourth sub-systems 122, 124, 126, and 128) respectively by each of the operation resources.

Each of the first, second, third, and fourth processor modules 112, 114, 116, and 118 may operate movement of particles, for the molecular dynamics simulation, using an operation algorithm (e.g., an operation process) and may divide and process an operation on a sub-system in the three-dimensional system 120. For example, the first processor module 112 may perform an operation on the first sub-system 126, the second processor module 114 may perform an operation on the second sub-system 128, the third processor module 116 may perform an operation on the third sub-system 122, and the fourth processor module 118 may perform an operation on the fourth sub-system 124. Processor modules other than the first, second, third, and fourth processor modules 112, 114, 116, and 118 may perform operations on the remaining sub-systems other than the first, second, third, and fourth sub-systems 122, 124, 126, and 128. The first, second, third, and fourth processor modules 112, 114, 116, and 118 may respectively perform an operation on movement of particles in corresponding sub-systems (e.g., the first, second, third, and fourth sub-systems 126, 128, 122, and 124) and perform communication in a mutually synchronized manner when information exchange is to be implemented in a border region among the first, second, third, and fourth sub-systems 122, 124, 126, and 128.

A plurality of processor modules may exchange an intermediate operation result through communication to calculate movement of a particle positioned in the border region among the first, second, third, and fourth sub-systems 122, 124, 126, and 128 or adjacent to the border region. This exchange of the intermediate operation result may be for an exchange of information on a neighboring particle because there may be information (e.g., a position, an applied force, speed) on the neighboring particle that is to be used for calculating the movement of the particle in another processor module. For example, to calculate the movement of a particle positioned in a border region between the first sub-system 126 and the second sub-system 128, the first processor module 112 and the second processor module 114 may be synchronized and share an operation result of another particle. Communication between the first processor module 112 and the second processor module 114 may be performed in synchronization with each other, and such communication between processor modules may have a great impact on time and speed of parallel processing. The communication between processor modules may sometimes generate a high communication overhead, and the performance speed of an entire communication process may be determined in accordance with communication efficiency between the processor modules.

As described above, when multiple operation resources perform a single task, the operation resources may be synchronized and intermediate data may be exchanged. Therefore, in large-scale parallel processing, efficient communication between the operation resources may have a great impact on overall performance. In an HPC application, communication data may generally have a certain pattern rather than a random feature, and the efficient communication between the operation resources may be achieved using a feature having the certain pattern. In examples described herein, various apparatuses and methods of one or more embodiments of performing efficient communication between processor modules are detailed, for example, by performing data prediction using a communication data pattern and thereby reducing the number of data transmissions and receptions and/or by transmitting compressed data and thereby reducing an amount of data transmissions. Such apparatuses and methods of one or more embodiments may improve the performance and speed of parallel processing. Hereinafter, non-limiting examples will be described in detail.

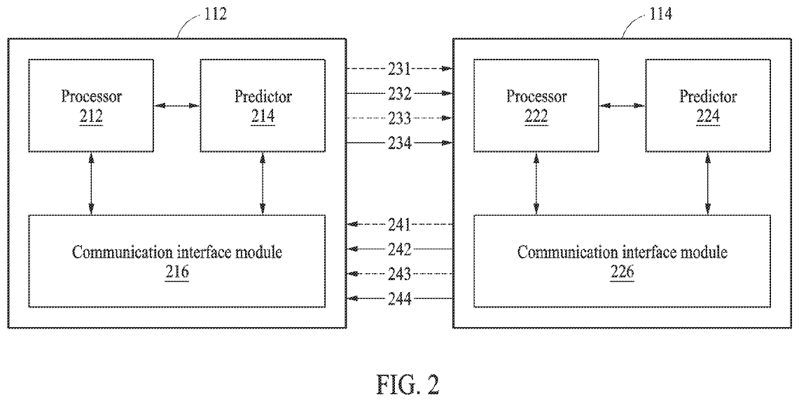

FIG. 2 illustrates an example of communication between processor modules.

Communication between the first processor module 112 and the second processor module 114 will be described as an example with reference to FIG. 2. The first processor module 112 and the second processor module 114 may perform parallel processing in synchronization with each other. The first processor module 112 may include a processor 212 (e.g., one or more processors), a predictor 214, and a communication interface module (e.g., a communication interface) 216, and the second processor module 114 may include a processor 222 (e.g., one or more processors), a predictor 224, and a communication interface module (e.g., a communication interface) 226. The first processor module 112 and the second processor module 114 may each further include a memory storing instructions that, when executed by the respective processors 212 and 222, configure the respective processors 212 and 222 to perform any one or more or all of the methods and operations of the respective processors 212 and 222 described herein.

The processors 212 and 222 may respectively control an overall operation of the first processor module 112 and the second processor module 114 and perform an operation process according to a defined operation algorithm. The processors 212 and 222 may be a CPU, a GPU, an NPU, and/or an FPGA, for example. The processors 212 and 222 may perform various data processing and/or an operation by executing software, for example, a molecular dynamics simulation. The predictors 214 and 224 may perform data prediction according to a prediction algorithm (e.g., a prediction process), and the communication interface modules 216 and 226 may communicate with another processor module. The communication interface modules 216 and 226 may exchange data with the other processor module through a communication link, for example, NVLink.

The processor 212 may determine first operation result data using the operation algorithm in a first time interval. For example, the processor 212 may determine the first operation result data by calculating position and movement information of a particle positioned in a border region between the first sub-system 126 and the second sub-system 128 or a border adjacent region in the first time interval according to a molecular dynamics simulation program.

The processor 212 may determine whether to transmit the determined first operation result data to the second processor module 114. The processor 212 may determine whether to transmit the first operation result data according to a defined transmission policy. For example, the processor 212 may determine to transmit the first operation result data when a current number of transmissions satisfies a set condition, a difference between the first operation result data and previous (e.g., previously determined) operation result data is greater than or equal to a threshold, and/or a difference between the first operation result data and a first prediction result data determined by the predictor 214 is greater than or equal to the threshold or another threshold. Operation result data may be transmitted once every n times (here, n is an integer greater than or equal to 2) according to the transmission policy and/or be transmitted when an error corresponding to a difference between the operation result data and prediction result data is greater than or equal to the threshold. Alternatively, the processor 212 may measure an average error based on the difference between the operation result data and the prediction result data and determine to transmit the operation result data when the average error is out of a certain range. The transmission policy may not be limited to the foregoing examples and be variously modified.

The predictor 214 may determine the first prediction result data using the prediction algorithm predicting current or future data based on previous data. The prediction algorithm may be, for example, an algorithm for determining the prediction result data using a Taylor series approximation technique approximating time series data with a polynomial function. However, the prediction algorithm may not be limited to using the Taylor series approximation technique, and any algorithm that may perform data prediction on the time series data may be applicable without limitations. The processor 212 may determine not to transmit (or skip the transmission of) the first operation result data when a transmission condition for the first operation result data is not satisfied. Hereinafter, skipping transmission of the operation result data may be replaced by, or include, transmitting a notification signal to notify the skipping of the transmission of the operation result data or a control signal to instruct to perform data prediction. The communication interface module 216 may transmit the first operation result data to the second processor module 114 when the transmission condition for the first operation result data is satisfied, and thereby the processor 212 determines to transmit the first operation result data to the second processor module 114. The second processor module 114 may receive the first operation result data from the first processor module 112 through the communication interface module 226.

The processor 212 may determine second operation result data using the operation algorithm in a second time interval, which is a time interval following the first time interval. The first operation result data and the second operation result data may correspond to the time series data. The processor 212 may determine whether to transmit the determined second operation result data to the second processor module 114. Similar to the foregoing example, the processor 212 may determine whether to transmit the second operation result data according to the defined transmission policy. When the processor 212 determines to transmit the first operation result data to the second processor module 114 and not to transmit the second operation result data to the second processor module 114, the second processor module 114 may predict the second operation result data using the predictor 224. The predictor 224 may determine second prediction result data corresponding to the second operation result data using the same prediction algorithm used by the predictor 214. The processor 222 may perform an operation in a next time interval using the second prediction result data.

As described above, the processor 212 may continuously generate operation result data (e.g., the first operation result data, the second operation result data, third operation result data, etc.) over the course of time based on the operation algorithm. The operation result data may be data to be used for an operation by the second processor module 114. The processor 212 may determine to transmit the generated operation result data only in a certain case rather than all the time. For example, the processor 212 may determine to transmit the operation result data in time intervals 232 and 234 and not to transmit the operation result data in time intervals 231 and 233. Such a transmission policy of one or more embodiments may reduce a data transmission number of times in an entire operation process, and thus, may increase processing speed.

When the processor 212 determines to transmit the second operation result data to the second processor module 114, the processor 212 may compress data to be transmitted to reduce a data transmission amount. For example, the processor 212 may compress difference data between the second operation result data and the first operation result data and transmit the compressed difference data to the second processor module 114 (e.g., instead of transmitting the entirety of the second operation result data). When receiving the compressed difference data from the first processor module 112, the second processor module 114 may decompress the compressed difference data, determine decompressed difference data, and then perform data restoration based on the decompressed difference data to restore the second operation result data. The second processor module 114 may restore the second operation result data based on the first operation result data and the decompressed difference data.

The operation result data (e.g., the first operation result data and the second operation result data) generated by the processor 212 may be data in a floating point type. In this case, the processor 212 may transmit an exponent part of the operation result data only once, for example, initially or at a time of change, to the second processor module 114 to reduce a data transmission amount, and thereafter transmit only a mantissa part excluding the exponent part of the data.

Similar to the first processor module 112, the second processor module 114 may generate operation result data to be used by the first processor module 112 based on the operation algorithm and transmit the generated operation result data to the first processor module 112. The processor 222 may determine to transmit the operation result data generated based on the operation algorithm only in a certain case rather than all the time. For example, the processor 222 may determine to transmit the operation result data in time intervals 242 and 244 and not to transmit the operation result data (or to skip data transmission) in time intervals 241 and 243.

A communication method of one or more embodiments between the first and second processor modules 112 and 114 described above may reduce a communication number of times and a size of data to be transmitted while allowing parallel communication. This may reduce processing time and improve performance. Reducing the number of communications by the skipping of the data transmission may effectively reduce an overall communication overhead since the first and second processor modules 112 and 114 operate in synchronization with each other. In addition, determining operation result data using the operation algorithm by the first processor module 112 and determining operation result data in a next time interval using the prediction algorithm by the second processor module 114 may be performed simultaneously in parallel. Accordingly, execution (or an operation) may be parallelized, and execution speed and processing efficiency of an entire process may be improved.

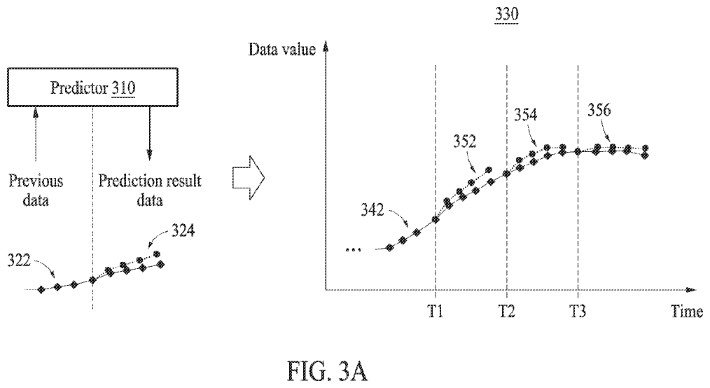

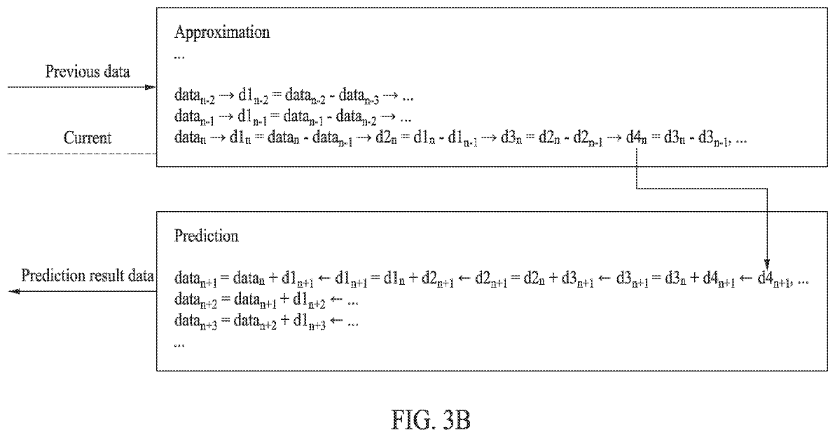

FIGS. 3A and 3B illustrate an example of a predictor.

Referring to FIG. 3A, operation result data transmitted between processor modules (e.g., the first processor module 112 and the second processor module 114 of FIG. 1) may be time series data and have a certain data pattern on a time axis. For example, the operation result data may have a value smaller as an order of a delta value (or a derivative value) increases. Thus, the operation result data may be approximated (or fitted) with a polynomial function, and future data may be predicted using previous data.

A predictor 310 (e.g., either one or both of the predictors 214 and 224 of FIG. 2) may approximate such operation result data as described above with a polynomial function and generate prediction result data from previous data using a prediction algorithm for performing data prediction. The prediction algorithm may be based on, for example, a Taylor series approximation technique, but an example may not be limited thereto. When there is operation result data 322 generated based on an operation algorithm, the predictor 310 may generate prediction result data 324 corresponding to operation result data at a certain time (e.g., a time subsequent to a time of the operation result data 322) based on previous operation result data (e.g., the operation result data 322). Since operation result data transmitted between processor modules may have a predictable pattern, a processor module on a transmitting side may not transmit the operation result data to a processor module on a receiving side. The processor module on the receiving side may generate prediction result data corresponding to the operation result data which is not transmitted through the predictor 310 and perform an operation thereafter using the generated prediction result data. The predictor 310 may generate prediction result data in the present or future using a data pattern of prediction result data generated in the past and/or operation result data received in the past.

A graph 330 may represent an example of a change in data values of operation result data 342 and prediction result data 352, 354, and 356 over the course of time. The operation result data 342 (illustrated by a solid line) may represent operation result data generated by the processor module on the transmitting side based on the operation algorithm. The prediction result data 352, 354, and 356 (illustrated by a dotted line) may represent prediction result data generated through the predictor 310 using the prediction algorithm by the processor module on the receiving side. Referring to the graph 330, the processor module on the transmitting side may synchronize a data value by transmitting operation result data to the processor module on the receiving side at times T1, T2, and T3, and the processor module on the receiving side may generate prediction result data corresponding to the operation result data using the predictor 310 during four time intervals (or time steps) after receiving the operation result data. The predictor 310 may generate a data value, which is not received as the prediction result data, by applying operation result data received in the past and/or prediction result data generated in the past to a polynomial function.

In an example, the processor module on the transmitting side may determine prediction result data based on the same prediction algorithm used by the processor module on the receiving side and determine whether to transmit operation result data based on a difference (or an error) between the operation result data and prediction result data corresponding thereto. When the difference is acceptable (e.g., when the difference is less than or equal to a certain threshold, for example), the processor module on the transmitting side may induce the processor module on the receiving side to generate the prediction result data by not transmitting the operation result data. When the difference is not acceptable, the operation result data may be transmitted to the processor module on the receiving side.

As described above, the processor module of one or more embodiments on the transmitting side may greatly reduce execution time of an entire operation process by transmitting the operation result data intermittently or when a certain transmission condition is satisfied rather than in every time interval (or time step).

Referring to FIG. 3B, an example of a Taylor series approximation technique is illustrated as a prediction algorithm used by the predictor 310. In FIG. 3B, data, may be data in an nth time interval (or time step), and d1ₙ, may be a first derivative value or a first delta value (or 1^{st} order derivative/delta value) of the data and may be represented as d1ₙ=dataₙ−dataₙ₋₁. d2ₙ may be a second derivative value or a second delta value and be represented as d2ₙ=d1ₙ−d1ₙ₋₁. The predictor 310 may derive an approximation equation of a polynomial expression through the Taylor series approximation technique based on time series data in the past and determine prediction result data in the future based on the derived approximation equation. This example is approximated as d4ₙ₊₁=d4ₙ. Accuracy may be improved as an approximation order increases, but computational complexity may also increase. The predictor 310 may balance a trade-off between efficiency and accuracy of prediction by adjusting the approximation order (e.g., a derivative value or a delta value) in determining prediction result data. Such an approximation order may be adjusted according to an error corresponding to a difference between operation result data and prediction result data. For example, when the error is great, the approximation order may be increased to improve accuracy, and when the error is small, the approximation order may be decreased to continuously reduce complexity.

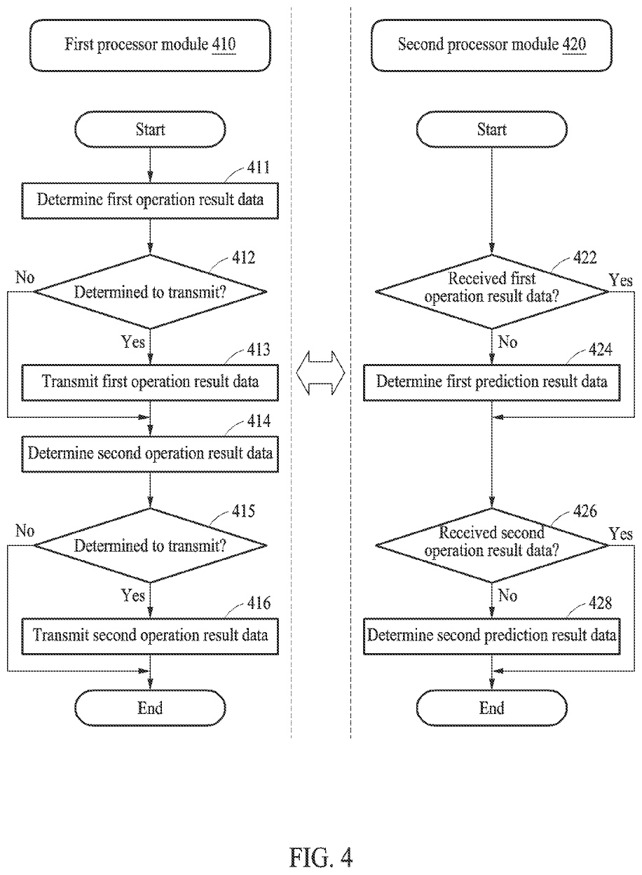

FIG. 4 illustrates an example of a communication method between processor modules in a parallel processing method.

Referring to FIG. 4, a first processor module 410 (e.g., the first processor module 112 of FIG. 2) and a second processor module 420 (e.g., the second processor module 114 of FIG. 2) may communicate with each other when performing parallel processing. In an example, the first processor module 410 may be a transmitting side for transmitting operation result data, and the second processor module 420 may be a receiving side for receiving the operation result data generated by the first processor module 410. The first processor module 410 may reduce a data transmission amount by transmitting operation result data to be used for prediction in the future only to the second processor module 420. In this case, the first processor module 410 may only transmit parameters (e.g., derivative values or delta values of an approximation equation) for a prediction algorithm rather than transmitting all the operation result data.

In operation 411, the first processor module 410 may determine first operation result data using an operation algorithm in a first time interval. The first processor module 410 may determine the first operation result data, for example, on movement of a particle using the operation algorithm for operating the movement of the particle over time for a molecular dynamics simulation.

In operation 412, the first processor module 410 may determine whether to transmit the first operation result data to the second processor module 420. The first processor module 410 may determine whether to transmit the first operation result data according to a defined transmission policy. For example, the first processor module 410 may determine to transmit the first operation result data when a current number of transmissions satisfies a defined condition (for example, whether the number is a multiple of n, where n is an integer greater than or equal to 2), a difference between the first operation result data and previous (e.g., previously determined) operation result data is greater than or equal to a threshold, and/or a difference (or a prediction error) between the first operation result data and first prediction result data predicted by a predictor is greater than or equal to the threshold or another threshold. Such a transmission policy may be shared in advance between the first processor module 410 and the second processor module 420. In operation 413, when determining to transmit the first operation result data, the first processor module 410 may transmit the first operation result data to the second processor module 420. When determining not to transmit the first operation result data, the first processor module 410 may skip transmission of the first operation result data. In addition, a transmission policy on operation result data may be variously set and not be limited to the foregoing example.

In operation 422, the second processor module 420 may determine whether the first operation result data is received from the first processor module 410. When the first operation result data is received, the second processor module 420 may store the received first operation result data. When the first processor module 410 determines not to transmit the first operation result data to the second processor module 420, and thus, when the second processor module 420 does not receive the first operation result data, in operation 424, the second processor module 420 may determine the first prediction result data corresponding to the first operation result data using the prediction algorithm. Data may be restored through prediction using the prediction algorithm.

In operation 414, the first processor module 410 may determine the second operation result data using the operation algorithm in a second time interval. In operation 415, the first processor module 410 may determine whether to transmit second operation result data to the second processor module 420. When determining to transmit the second operation result data, in operation 416, the first processor module 410 may transmit the second operation result data to the second processor module 420. When determining not to transmit the second operation result data, the first processor module 410 may skip transmission of the second operation result data.

In an example, in operation 415, the first processor module 410 may determine not to transmit the second operation result data to the second processor module 420 when a difference between the second operation result data and the first operation result data is 0 or less than a threshold. When the difference is greater than or equal to the threshold, the first processor module 410 may determine to transmit the second operation result data to the second processor module 420.

In another example, in operation 415, the first processor module 410 may determine to transmit the second operation result data to the second processor module 420 when a current number of times of transmission to the second processor module 420 satisfies a set condition (for example, the number is a multiple of n, where n is an integer greater than or equal to 2). When the current number of transmissions does not satisfy the set condition, the transmission of the second operation result data may be skipped.

In yet another example, in operation 415, the first processor module 410 may determine the second prediction result data based on the first operation result data and the prediction algorithm, and when a difference between the second operation result data and the second prediction result data determined by the first processor module 410 is less than the threshold, determine not to transmit the second operation result data to the second processor module 420. When the difference is greater than or equal to the threshold, the first processor module 410 may determine to transmit the second operation result data to the second processor module 420.

The first operation result data and the second operation result data may be data in a floating point type. In this case, in operation 413, the first processor module 410 may transmit an exponent part of the first operation result data initially or at a time of change and thereafter only transmit a mantissa part excluding the exponent part of the data to the second processor module 420. In operation 416, the first processor module 410 may transmit a mantissa part excluding the exponent part of the second operation result data to the second processor module 420.

In operation 426, the second processor module 420 may determine whether the second operation result data is received from the first processor module 410. When the second operation result data is received, the second processor module 420 may store the received second operation result data. When the first processor module 410 determines not to transmit the second operation result data to the second processor module 420, and thus, when the second processor module 420 does not receive the second operation result data, in operation 428, the second processor module 420 may determine the second prediction result data corresponding to the second operation result data based on the first operation result data (or the first prediction result data) and the prediction algorithm. Here, the second processor module 420 may determine whether the second prediction result data is determinable based on the received first operation result data and the prediction algorithm, and when the second prediction result data is determined to be determinable, may determine the second prediction result data.

The first processor module 410 and the second processor module 420 may perform such a process described above continuously in time intervals after the second time interval. Skipping transmission of operation result data and predicting and using prediction result data may reduce a data synchronization process between the first and second processor modules 410 and 420, and processing speed may thus be increased.

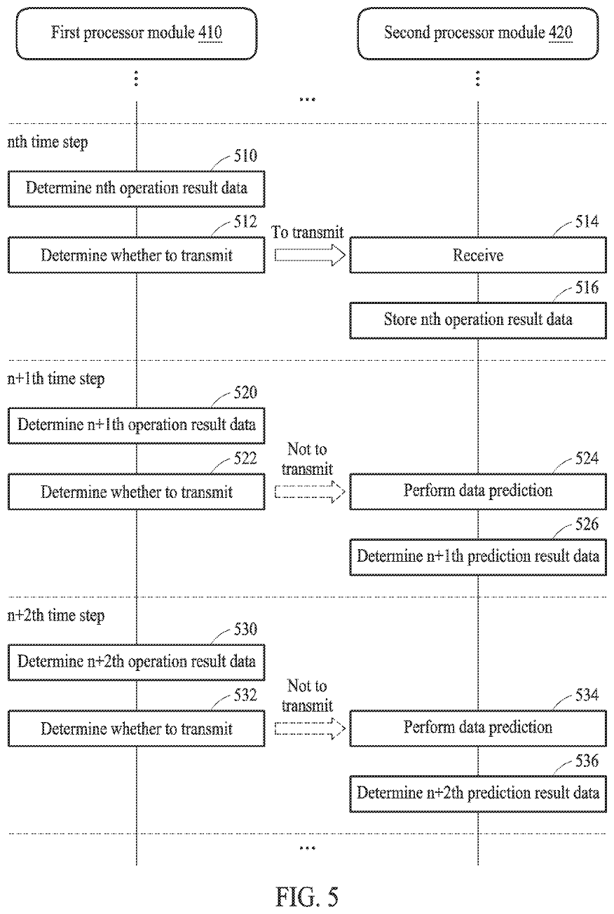

FIG. 5 illustrates an example of communication between processor modules.

Referring to FIG. 5, operations 510, 512, 514, and 516 may be performed in an nth time step. The first processor module 410 may determine nth operation result data based on an operation algorithm in operation 510, and in operation 512, may determine whether to transmit the nth operation result data. The first processor module 410 may determine whether to transmit the nth operation result data according to whether a current number of transmissions satisfies a defined transmission policy. When the transmission policy is to transmit operation result data when the current number of transmissions corresponds to a multiple of 3, and when the current number of transmissions is a multiple of 3, the first processor module 410 may transmit the nth operation result data to the second processor module 420. The second processor module 420 may receive the nth operation result data from the first processor module 410 in operation 514, and in operation 516, may store the nth operation result data.

In an n+1th time step, or the next time step of the nth time step, operations 520, 522, 524, and 526 may be performed. The first processor module 410 may determine n+1th operation result data based on the operation algorithm in operation 520, and may determine whether to transmit the n+1th operation result data in operation 522. When the current number of transmissions (e.g., 4) does not satisfy a defined condition (e.g., does not correspond to a multiple of 3), the first processor module 410 may determine not to transmit the n+1th operation result data to the second processor module 420. In this case, the second processor module 420 may perform data prediction in operation 524 using previous data (e.g., the nth operation result data) and a prediction algorithm. Through the data prediction, the second processor module 420 may determine n+1th prediction result data corresponding to the n+1th operation result data in operation 526.

In an n+2th time step, or the next time step of the n+1th time step, operations 530, 532, 534, and 536 may be performed. Operations 530, 532, 534, and 536 in the n+2th time step may be similar to operations 520, 522, 524, and 526 in the n+1th time step, and a repeated description thereof is omitted. In an n+3th time step, the current number of transmissions may correspond to a multiple of 3 again, and thus, the first processor module 410 may transmit n+3th operation result data to the second processor module 420. As described above, transmission of operation result data between the first processor module 410 and the second processor module 420 may be performed periodically and repeatedly according to the defined transmission policy.

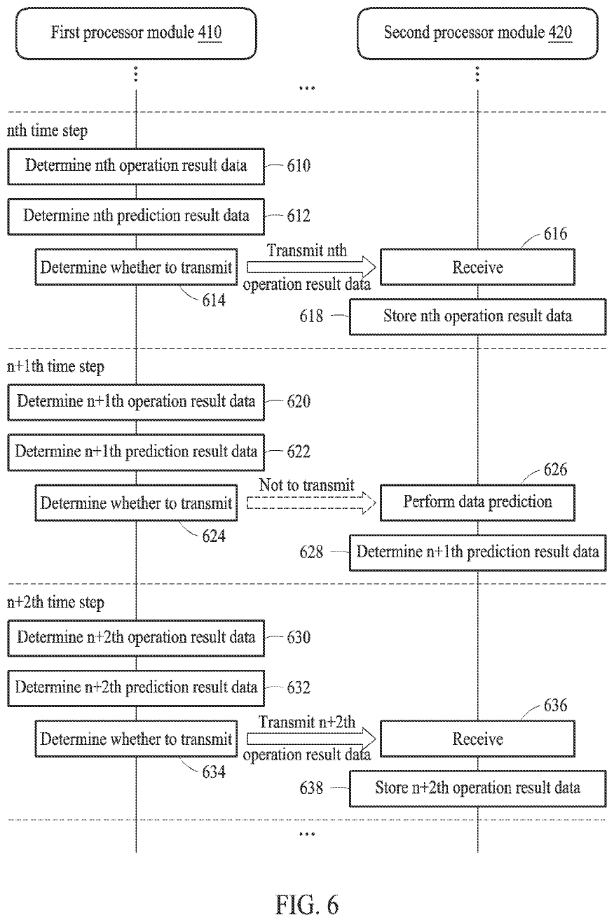

FIG. 6 illustrates another example of communication between processor modules.

Referring to FIG. 6, operations 610, 612, 614, 616, and 618 may be performed in an nth time step. The first processor module 410 may determine nth operation result data based on an operation algorithm in operation 610, and in operation 612, may determine nth prediction result data based on a prediction algorithm. In operation 614, the first processor module 410 may determine whether to transmit the nth operation result data based on the nth operation result data and the nth prediction result data. For example, the first processor module 410 may determine to transmit the nth operation result data when a difference between the nth operation result data and the nth prediction result data is greater than or equal to a threshold, and when the difference is less than the threshold, may determine not to transmit the nth operation result data. In this example, when the difference between the nth operation result data and the nth prediction result data is greater than or equal to the threshold, the first processor module 410 may transmit the nth operation result data to the second processor module 420. The second processor module 420 may receive the nth operation result data from the first processor module 410 in operation 616, and in operation 618, may store the nth operation result data.

In an n+1th time step, or the next time step of the nth time step, operations 620, 622, 624, 626 and 628 may be performed. The first processor module 410 may determine n+1th operation result data based on an operation algorithm in operation 620, and may determine n+1th prediction result data based on a prediction algorithm in operation 622. In operation 624, the first processor module 410 may determine whether to transmit the n+1th operation result data based on the n+1th operation result data and the n+1th prediction result data. When a difference between the n+1th operation result data and the n+1th prediction result data is less than the threshold, the first processor module 410 may not transmit the n+1th operation result data to the second processor module 420. In this case, the second processor module 420 may perform data prediction using previous data (e.g., the nth operation result data) and a prediction algorithm in operation 626, and may determine the n+1th prediction result data corresponding to the n+1th operation result data in operation 628. Here, the prediction algorithm used for the data prediction by the second processor module 420 may be the same prediction algorithm used in operation 622 by the first processor module 410.

In an n+2th time step, or the next time step of the n+1th time step, operations 630, 632, 634, 636, and 638 may be performed. Operations 630, 632, 634, 636, and 638 in the n+2th time step may be similar to operations 610, 612, 614, 616, and 618 in the nth time step, and a repeated description thereof is omitted. In the nth time step and the n+2th time step, operation result data determined by the first processor module 410 may be transmitted to the second processor module 420, and data synchronization may be performed.

As described above, the first processor module 410 may determine in advance an error to be expected when the second processor module 420 generates prediction result data through a prediction algorithm, and when the error is less than a threshold, may determine to skip transmission of operation result data. A range of the error included in the prediction result data generated by the second processor module 420 through data prediction may thus be limited within a certain range.

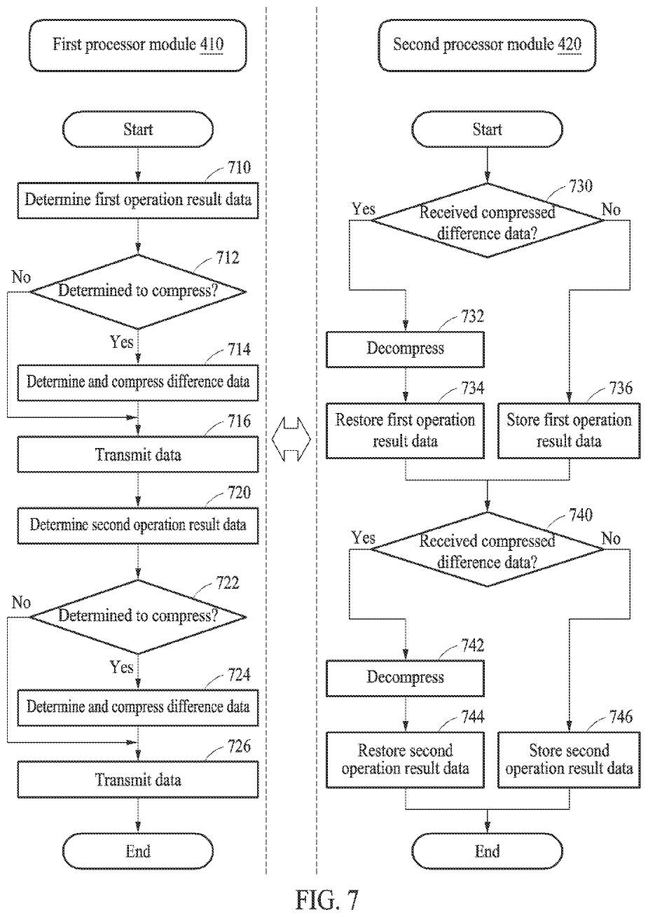

FIG. 7 illustrates another example of a communication method between processor modules in a parallel processing method.

Referring to FIG. 7, in operation 710, the first processor module 410 may determine first operation result data based on an operation algorithm. In operation 712, the first processor module 410 may determine whether to compress data. For example, the first processor module 410 may determine not to perform data compression when an error to be expected as a result of the data compression is greater than or equal to a threshold, and when the error is less than the threshold, may determine to perform data compression. The first processor module 410 may determine to transmit original data of the first operation result data when compression may not be possible in such a case as initial transmission, and when the compression is possible, may determine to perform data compression. When determining not to compress data, the first processor module 410 may transmit uncompressed first operation result data to the second processor module 420.

When determining to compress data, in operation 714, the first processor module 410 may determine difference data representing a difference between the first operation result data and previous operation result data of the first operation result data and may compress the difference data. Then, in operation 716, the first processor module 410 may transmit the compressed difference data to the second processor module 420. All data other than difference data may be transmitted at least once before the compressed difference data is transmitted. As a compression scheme, the first processor module 410 may not transmit a zero bit included in the difference data and only transmit a bit other than the zero bit and may thereby maintain data accuracy while reducing a data transmission amount. As another example, the first processor module 410 may reduce a data size by changing a data type (e.g., double (8bytes), float (4 bytes), and half (2 bytes)). As yet another example, the first processor module 410 may reduce a data transmission amount by omitting transmission of an exponent part of data consecutively having the same value and by only transmitting a mantissa part of data when transmitting difference data in a floating point type. The first processor module 410 may increase a compression ratio by increasing a compression dimension of difference data. A method of performing data compression is not limited to the foregoing examples, and various other data compression techniques may be applied to compress difference data.

In operation 730, the second processor module 420 may determine whether to receive the compressed difference data from the first processor module 410. When the compressed difference data is received, in operation 732, the second processor module 420 may decompress the compressed difference data and determine decompressed difference data. In operation 734, the second processor module 420 may perform data restoration on the first operation result data based on the decompressed difference data. The second processor module 420 may restore the first operation result data by applying the decompressed difference data to the previous operation result data of the first operation result data. When the first operation result data other than the compressed difference data is received from the first processor module 410, in operation 736, the second processor module 420 may store the received first operation result data.

In operation 720, the first processor module 410 may determine second operation result data based on the operation algorithm. In operation 722, the first processor module 410 may determine whether to compress data. When determining not to compress data, the first processor module 410 may transmit uncompressed second operation result data to the second processor module 420. When determining to compress data, in operation 724, the first processor module 410 may determine difference data representing a difference between the second operation result data and the first operation result data and may compress the difference data. In operation 726, the first processor module 410 may transmit the compressed difference data to the second processor module 420.

In operation 740, the second processor module 420 may determine whether to receive the compressed difference data from the first processor module 410. When the compressed difference data is received, in operation 742, the second processor module 420 may decompress the compressed difference data and determine decompressed difference data. In operation 744, the second processor module 420 may perform data restoration on the second operation result data based on the decompressed difference data. The second processor module 420 may restore the second operation result data by applying the first operation result data and the decompressed difference data. When the second operation result data other than the compressed difference data is received from the first processor module 410, in operation 746, the second processor module 420 may store the received second operation result data.

The first processor module 410 and the second processor module 420 may perform such a process described above continuously in time intervals thereafter. Compressing and transmitting transmission data may reduce a communication overhead, and thereby data transmission speed may be increased, and processing speed may be increased. More particularly, compressing and transmitting only difference data different from previous operation result data may prevent repeated transmission of redundant data in data transmission.

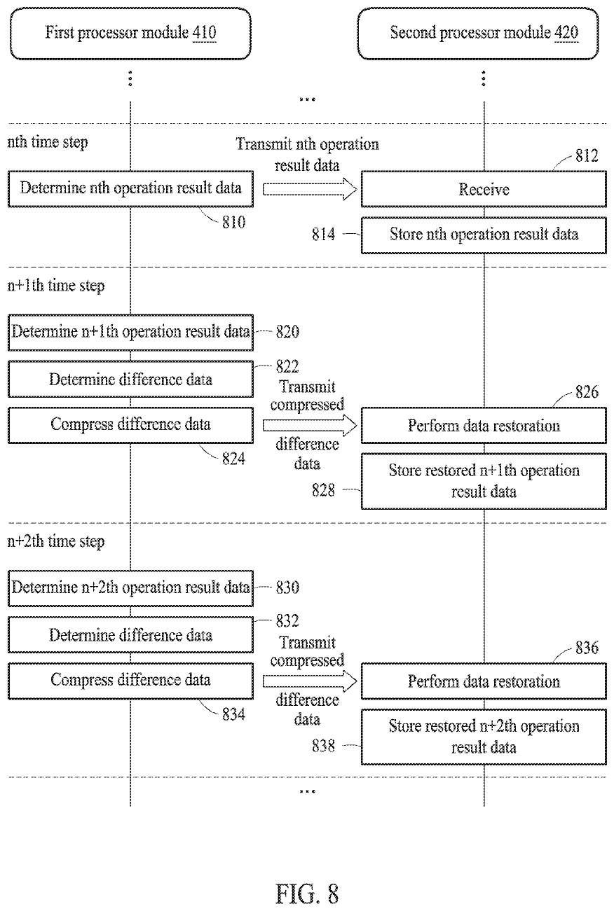

FIG. 8 illustrates yet another example of communication between processor modules.

Referring to FIG. 8, operations 810, 812, and 814 may be performed in an nth time step. In operation 810, the first processor module 410 may determine nth operation result data based on an operation algorithm. The first processor module 410 may transmit all data of the determined nth operation result data to the second processor module 420. The second processor module 420 may receive the nth operation result data from the first processor module 410 in operation 812, and in operation 814, may store the nth operation result data.

In an n+1th time step, or the next time step of the nth time step, operations 820, 822, 824, 826, and 828 may be performed. In operation 820, the first processor module 410 may determine n+1th operation result data based on the operation algorithm. The first processor module 410 may determine difference data between the n+1th operation result data and the nth operation result data in operation 822, and in operation 824, may compress the difference data. The first processor module 410 may transmit the compressed difference data to the second processor module 420, and the second processor module 420 may receive the compressed difference data from the first processor module 410. In operation 826, the second processor module 420 may decompress the compressed difference data, determine decompressed difference data, perform data restoration based on the decompressed difference data and the previously received nth operation result data, and restore the n+1th operation result data. The n+1th operation result data may be restored by adding the decompressed difference data to the nth operation result data. In operation 828, the second processor module 420 may store the restored n+1th operation result data.

In an n+2th time step, or the next time step of the n+1th time step, operations 830, 832, 834, 836, and 838 may be performed. Operations 830, 832, 834, 836, and 838 in the n+2th time step may be similar to operations 820, 822, 824, 826, and 828 in the n+1th time step, and a repeated description thereof is omitted. In the n+2th time step, difference data between n+2th operation result data and the n+1th operation result data may be compressed and transmitted to the second processor module 420. The second processor module 420 may restore the n+2th operation result data based on the compressed difference data and the n+1th operation result data. The second processor module 420 may decompress the compressed difference data and restore the n+2th operation result data by adding the decompressed difference data to the n+1th operation result data.

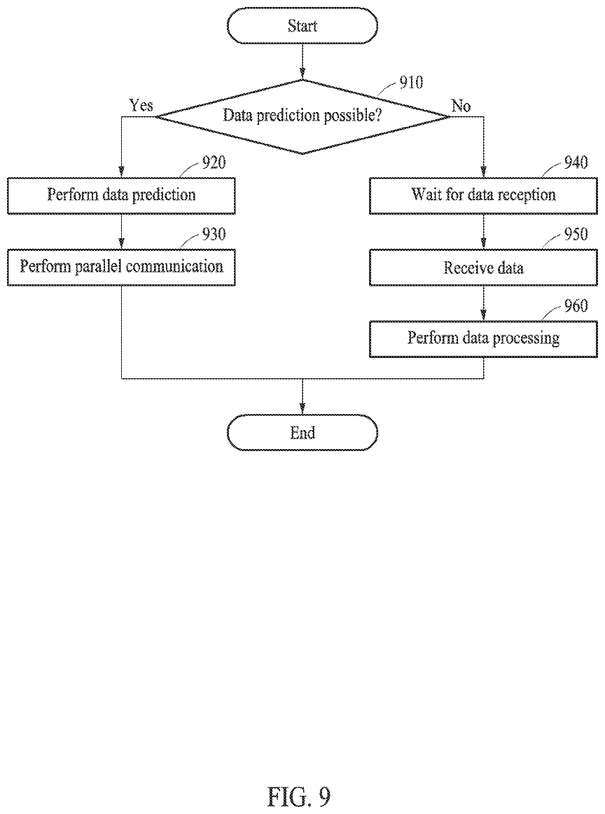

FIG. 9 illustrates still another example of a communication method between processor modules in a parallel processing method.

Referring to FIG. 9, in operation 910, a second processor module (e.g., the second processor module 420 of FIG. 4) may determine whether data prediction for determining prediction result data based on previous data is possible. The second processor module may determine whether there is dependency between the previous data and data to be predicted. When there is no dependency, the second processor module may determine that the data prediction is possible. That there is dependency may mean that there is data that may need to be received in advance from another processor module to accurately transmit certain data.

When determining that data prediction is possible, in operation 920, the second processor module may perform the data prediction using a prediction algorithm. Prediction result data may be generated as a result of performing data prediction. For example, when the second processor module previously receives first operation result data from a first processor module, the second processor module may determine second prediction result data based on the first operation result data and the prediction algorithm.

In operation 930, the second processor module may perform parallel communication. For example, the second processor module may receive operation result data from the first processor module (e.g., the first processor module 410 of FIG. 4), and at the same time, transmit prediction result data to another processor module (e.g., the third processor module 116 of FIG. 1). In addition, the second processor module may receive second operation result data from the first processor module and transmit the second prediction result data to another processor module.

In an example, determining operation result data using an operation algorithm by the first processor module and determining operation result data in a next time interval using the prediction algorithm by the second processor module may be performed simultaneously in parallel. For example, receiving the second operation result data from the first processor module by the second processor module and transmitting the second prediction result data to another processor module may be performed simultaneously in parallel. The apparatus and method of one or more embodiments may thereby transform sequential communication of data transmission into concurrent communication, and the apparatus and method of one or more embodiments may thereby further increase overall processing speed. An execution operation other than such a transmission operation may be simultaneously performed. Accordingly, execution (or an operation) may be parallelized, and the apparatus and method of one or more embodiments may thus improve execution speed and processing efficiency of an entire process. When there is dependency between operation processing processes, a result value of a previous operation may typically be determined (or prepared) to execute (or operate) a next operation in an entire operation process, which may only be “executed sequentially”. However, as the second processor module may predict and determine in advance a result value of a next operation through data prediction without the result value of the previous operation, the dependency between the operation processing processes may be removed, and the operation processing processes may be “executed simultaneously in parallel” rather than being executed sequentially. Thus, the apparatus and method of one or more embodiments may increase execution speed of an entire process. In addition, an operation device may not be effectively used due to a waiting process involved in typical sequential execution. In contrast, the apparatus and method of one or more embodiments may perform an execution process simultaneously in parallel, and thus may increase overall operation efficiency by allowing effective use of the operation device, and thus, may perform an operation significantly faster even when using the same operation device or the same resource (e.g., power). When there is no dependency, the second processor module may generate the second prediction result data and prediction result data thereafter and transmit the generated prediction result data through parallel communication.

When data prediction is determined not to be possible in operation 910, the second processor module, in operation 940, may wait until data (e.g., operation result data or difference data) is received from the first processor module. When, in operation 950, the second processor module receives the data from the first processor module, the second processor module, in operation 960, may perform data processing based on the received data. For example, the second processor module may perform an operation based on operation result data received from the first processor module or perform data restoration based on difference data received from the first processor module.

The parallel processing apparatuses, first processor modules, second processor modules, third processor modules, fourth processor modules, processors, predictors, communication interface modules, processors, predictors, parallel processing apparatus 100, first processor module 112, second processor module 114, third processor module 116, fourth processor module 118, processor 212, predictor 214, communication interface module 216, processor 222, predictor 224, communication interface module 226, predictor 310, and other devices, apparatuses, units, modules, and components described herein with respect to FIG. 1-9 are implemented by or representative of hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-9 that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions in the specification, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

What is claimed is:

1. An apparatus with parallel processing, the apparatus comprising:
a first processor module; and
a second processor module configured to perform parallel processing in synchronization with the first processor module,
wherein the first processor module is configured to:
determine first operation result data using an operation process in a first time interval;
transmit the first operation result data to the second processor module;
determine second operation result data using the operation process in a second time interval; and
determine whether to transmit the second operation result data to the second processor module, and
wherein the second processor module is configured to determine second prediction result data corresponding to the second operation result data based on the first operation result data received from the first processor module and a prediction process in response to the first processor module determining not to transmit the second operation result data to the second processor module.

2. The apparatus of claim 1, wherein, for the determining of whether to transmit the second operation result data, the first processor module is configured to determine not to transmit the second operation result data to the second processor module in response to a difference between the second operation result data and the first operation result data being less than a threshold.

3. The apparatus of claim 1, wherein, for the determining of whether to transmit the second operation result data, the first processor module is configured to:
determine the second prediction result data based on the first operation result data and the prediction process; and
determine not to transmit the second operation result data to the second processor module in response to a difference between the second operation result data and the second prediction result data determined by the first processor module being less than a threshold.

4. The apparatus of claim 1, wherein, for the determining of whether to transmit the second operation result data, the first processor module is configured to determine to transmit the second operation result data to the second processor module in response to a current number of transmissions to the second processor module satisfying a set condition.

5. The apparatus of claim 1, wherein
the first operation result data and the second operation result data are data in a floating point type, and
the first processor module is configured to transmit a mantissa part excluding an exponent part of the second operation result data to the second processor module in response to determining to transmit the second operation result data to the second processor module.

6. The apparatus of claim 1, wherein
the first operation result data and the second operation result data correspond to time series data, and
the prediction process comprises determining prediction result data using a Taylor series approximation technique approximating the time series data with a polynomial function.

7. The apparatus of claim 1, wherein the first processor module is configured to:
determine whether to transmit the first operation result data to the second processor module; and
for the transmitting of the first operation result data, transmit the first operation result data to the second processor module in response to determining to transmit the first operation result data to the second processor module.

8. The apparatus of claim 1, wherein the second processor module is configured to:
determine whether the second prediction result data is determinable based on the received first operation result data and the prediction process and
for the determining of the second prediction result data, determine the second prediction result data in response to the second prediction result data being determined to be determinable.

9. The apparatus of claim 1, wherein, in response to determining to transmit the second operation result data to the second processor module, the first processor module is configured to compress difference data between the second operation result data and the first operation result data and transmit the compressed difference data to the second processor module.

10. The apparatus of claim 9, wherein the second processor module is configured to determine decompressed difference data by decompressing the compressed difference data and perform data restoration based on the decompressed difference data in response to receiving the compressed difference data from the first processor module.

11. The apparatus of claim 1, wherein the first processor module and the second processor module are configured to operate movement of particles for a molecular dynamics simulation using the operation process.

12. The apparatus of claim 1, wherein the determining of the first operation result data using the operation process by the first processor module and the determining of the second prediction result data in a next time interval using the prediction process by the second processor module are performed simultaneously in parallel.

13. A processor-implemented method with parallel processing, the method comprising:
determining first operation result data using an operation process by a first processor module in a first time interval;
transmitting the first operation result data to a second processor module by the first processor module;
determining second operation result data using the operation process by the first processor module in a second time interval;
determining whether to transmit the second operation result data to the second processor module by the first processor module; and
determining second prediction result data corresponding to the second operation result data based on the first operation result data received from the first processor module by the second processor module and a prediction process without transmitting the second operation result data to the second processor module by the first processor module in response to the first processor module determining not to transmit the second operation result data to the second processor module.

14. The method of claim 13, wherein the determining of whether to transmit the second operation result data comprises determining not to transmit the second operation result data to the second processor module in response to a difference between the second operation result data and the first operation result data being less than a threshold.

15. The method of claim 13, wherein the determining of whether to transmit the second operation result data comprises:
determining the second prediction result data based on the first operation result data and the prediction process by the first processor module; and
determining not to transmit the second operation result data to the second processor module in response to a difference between the second operation result data and the second prediction result data determined by the first processor module being less than a threshold.

16. The method of claim 13, wherein the determining of whether to transmit the second operation result data comprises determining to transmit the second operation result data to the second processor module in response to a current number of transmissions to the second processor module satisfying a set condition.

17. The method of claim 13, wherein
the first operation result data and the second operation result data correspond to time series data, and
the prediction process comprises determining prediction result data using a Taylor series approximation technique approximating the time series data with a polynomial function.

18. The method of claim 13, further comprising:
determining whether the second prediction result data is determinable based on the first operation result data and the prediction process by the second processor module,
wherein the determining of the second prediction result data by the second processor module comprises determining the second prediction result data in response to the second prediction result data being determined to be determinable.

19. The method of claim 13, further comprising:
determining whether to transmit the first operation result data to the second processor module by the first processor module, wherein the transmitting of the first operation result data by the first processor module comprises transmitting the first operation result data to the second processor module in response to determining to transmit the first operation result data to the second processor module.

20. The method of claim 13, further comprising:
transmitting the second operation result data to the second processor module by the first processor module and determining the second prediction result data based on the first operation result data received from the first processor module by the second processor module and the prediction process in response to the first processor module determining to transmit the second operation result data to the second processor module; and
receiving the second operation result data from the first processor module by the second processor module and transmitting the second prediction result data to another processor module,
wherein the receiving of the second operation result data from the first processor module by the second processor module and the transmitting of the second prediction result data to the other processor module are performed simultaneously in parallel.

21. A non-transitory computer-readable storage medium storing instructions that, when executed by one or more processors, configure the one or more processors to perform the method of claim 13.
